(19) United States
(12) Patent Application Publication
(10) Pub. No.: US 2025/0180542 A1
(43) Pub. Date: Jun. 5, 2025

(54) INDIVIDUALIZED PATIENT-DERIVED TUMOR ORGANOIDS

(71) Applicants: SHANGHAITECH UNIVERSITY, Shanghai (CN); HUASHAN HOSPITAL, FUDAN UNIVERSITY, Shanghai (CN)

(72) Inventors: Haikun LIU, Shanghai (CN); Xiujian MA, Shanghai (CN); Changwen WANG, Shanghai (CN); Yonghe WU, Shanghai (CN); Chunxuan SHAO, Shanghai (CN); Youjun CHU, Shanghai (CN); Tianping PENG, Shanghai (CN); Meng SUN, Shanghai (CN); Wei HUA, Shanghai (CN); Ying MAO, Shanghai (CN)

(21) Appl. No.: 18/836,710

(22) PCT Filed: Feb. 13, 2023
(86) PCT No.: PCT/CN2023/075746

§ 371 Date: Aug. 7, 2024

(30) Foreign Application Priority Data
Feb. 11, 2022 (WO) PCT/CN2022/075999

(51) Int. Cl. G01N 33/50; C12N 5/071; C12N 5/09

(52) U.S. Cl. CPC G01N 33/5011; C12N 5/0613; C12N 5/0693; C12N 5/0697; G01N 33/5082; C12N 2501/115; C12N 2501/727; C12N 2506/45

(57) ABSTRACT

The present disclosure provides compositions and methods for preparing and using individualized patient-derived tumor organoids (IPTO). Human iPSC-derived cerebral organoids can be used as hosts for tumor tissue grafts. Patient materials can be obtained directly after surgery and dissected into small pieces. The small tumor tissue is inserted into the cerebral organoid to prepare a hybrid organoid. This hybrid organoid can grow for several weeks to months, allowing even slow-growing tumors, such as glioma with IDH mutations and pilocytic astrocytoma, to proliferate. The tumor pieces preferably include both tumor cells and adjacent stromal cells, allowing testing of conventional chemotherapeutic agents as well as immunotherapeutic agents.

### BACKGROUND

Predicting drug response is one of the major bottlenecks to select the best drug candidate for clinical treatment in personalized cancer therapy. Personalized treatments require access to reliable patient materials for individual drug testing before performing a clinical treatment.

Patient-derived cancer cells (PDCs) and patient-derived xenografts (PDXs) are often used as tumor models, but each has many shortcomings. PDCs lack diversity in terms of cell type, spatial organization, and microenvironment. PDXs, on the other hand, have low transplantation success rates and require a long culture time.

More recently, organoids have been developed as a three-dimensional cell culture. An organoid is a cell mass constructed in vitro and can be generated from embryonic stem cells, induced pluripotent stem cells (iPSCs), or somatic stem cells (SSCs). With the capabilities to self-renew and proliferate, organoids can also maintain the physiological structure and function of their source tissues.

Several brain tumor organoid models have been deciphered. However, these models lack the original tissue architecture or the interactions between tumor cells and non-tumor cells, which represent the major limitations for brain tumor organoid models.

### SUMMARY

The present disclosure describes a new individualized patient-derived tumor organoid (IPTO) system that overcomes the limitations of the conventional tumor organoid systems. Human iPSC-derived cerebral organoids can be used as hosts for tumor tissue grafts. Patient materials can be obtained directly after surgery and dissected into small pieces. The small tumor tissue is inserted into the cerebral organoid to prepare a hybrid organoid. This hybrid organoid can grow from several weeks to months, allowing even slow-growing tumors such as glioma with IDH mutations and pilocytic astrocytoma to proliferate. The IPTO preferably includes both tumor cells and adjacent stromal cells, allowing testing of anticancer agents for both efficacy and safety.

In one embodiment, the present disclosure provides a hybrid organoid, comprising a tumor tissue embedded in a cerebral organoid.

In some embodiments, the cerebral organoid is differentiated from a stem cell. In some embodiments, the stem cell is an induced pluripotent stem cell (iPSC). In some embodiments, the cerebral organoid expresses at least a marker selected from the group consisting of neuroepithelial stem cell protein (nestin), doublecortin (DCX), neuron-specific Class III β-tubulin (TuJ1), microtubule associated protein 2 (MAP2), marker of proliferation Ki-67 (KI67), paired box 6 (PAX6), vimentin, and T-box brain transcription factor 1 (TBR1).

In some embodiments, the tumor tissue has a size of 0.2 mm to 5 mm in diameter when disposed in the cerebral organoid. In some embodiments, the tumor tissue has a size of 0.5 mm to 2 mm in diameter when disposed in the cerebral organoid.

In some embodiments, the tumor tissue is a tissue of brain tumor. In some embodiments, the brain tumor is selected from the group consisting of glioblastoma, pilocytic astrocytoma, oligodendroglioma, and a metastatic tumor originated from another tissue.

In some embodiments, some of the brain tumor is characterized with a mutation in an isocitrate dehydrogenase (IDH). In some embodiments, the tumor tissue comprises tumor cells and adjacent non-tumor stromal cells.

In some embodiments, the tumor tissue has grown at least 50% in tumor cell number as compared to when the tumor tissue was initially disposed in the cerebral organoid.

In some embodiments, the tumor tissue has grown at least 2-fold in tumor cell number as compared to when the tumor tissue was initially disposed in the cerebral organoid. In some embodiments, the tumor tissue has at least doubled in size as compared to when the tumor tissue was initially disposed in the cerebral organoid.

In some embodiments, the hybrid organoid expresses at least a marker selected from the group consisting platelet endothelial cell adhesion molecule (PECAM-1, CD31), protein tyrosine phosphatase, receptor type, C (PTPRC, CD45)), cluster of differentiation 68 (CD68), doublecortin (DCX), glial fibrillary acidic protein (GFAP), glycerol-3-phosphate dehydrogenase 1 (GPD1), allograft inflammatory factor 1 (Iba1), marker of proliferation Ki-67 (Ki67), microtubule associated protein 2 (MAP2), neuroepithelial stem cell protein (nestin), oligodendrocyte transcription factor (Olig2), S100beta chain, SRY (sex determining region Y)-box 2 (Sox2), and neuron-specific class III β-tubulin (TuJ1).

Also provided, in one embodiment, is a method of evaluating a candidate anticancer agent, comprising contacting the hybrid organoid of any one of claims 1-13 with the candidate anticancer agent, and examining change of cell numbers in the hybrid organoid.

In some embodiments, a decrease of live tumor cells in the hybrid organoid indicates efficacy of the anticancer agent. In some embodiments, a decrease of live host cells in the hybrid organoid indicates toxicity of the anticancer agent.

In some embodiments, the hybrid organoid has been cultured for 1 to 16 weeks since the tumor tissue was initially disposed in the cerebral organoid. In some embodiments, the hybrid organoid has been cultured for 1 to 3 weeks since the tumor tissue was initially disposed in the cerebral organoid. In some embodiments, the hybrid organoid has been cryopreserved and recovered.

Another embodiments provides a method for preparing a hybrid organoid of the present disclosure, comprising disposing a tumor tissue in an incision of a cerebral organoid.

In some embodiments, the method further comprises covering the hybrid organoid with Matrigel® solubilized basement membrane matrix, and allowing the matrix to solidify. In some embodiments, the tumor tissue has a size of 0.2 mm to 5 mm in diameter when disposed in the cerebral organoid. In some embodiments, the tumor tissue has a size of 0.5 mm to 2 mm in diameter when disposed in the cerebral organoid. In some embodiments, the tumor tissue is a tissue of brain tumor. In some embodiments, the brain tumor is selected from the group consisting of glioblastoma, pilocytic astrocytoma, oligodendroglioma, and a metastatic tumor originated from another tissue.

In some embodiments, the cerebral organoid is prepared from an induced pluripotent stem cell (iPSC). In some embodiments, the cerebral organoid expresses at least a marker selected from the group consisting of neuroepithelial stem cell protein (nestin), doublecortin (DCX), neuron-specific Class III β-tubulin (TuJ1), microtubule associated protein 2 (MAP2), marker of proliferation Ki-67 (KI67), paired box 6 (PAX6), vimentin, and T-box brain transcription factor 1 (TBR1).

In some embodiments, the method further comprises cryopreserving the hybrid organoid. In some embodiments, the cryopreservation is in a cryopreservation medium that comprises a ROCK inhibitor.

### BRIEF DESCRIPTION OF THE DRAWINGS

FIG. 1a is a schematic diagram illustrating the procedure to establish IPTO. FIG. 1b shows immunostaining results for various neural cell markers in host organoids.

FIG. 2a-e. Culturing IDHmut glioma and pilocytic astrocytoma in IPTO. a. Cell density of IDHmut glioma-derived IPTO was comparable to the parental tumor (H&E staining, left) and secretion of 2-HG from IDHmut glioma-derived IPTO was detectable. b. representative IHC (IDHR132H) images showed spatial distribution of IDHmut glioma cells in corresponding IPTO. c. Tumor cell density in pilocytic astrocytoma-derived IPTO was high, and proliferating cells (EdU+) in the tumor area (GFP-) were identified. d. Tumor cells were indicated with GFAP expression and lack of GFP expression. e. Tumor proliferates as indicated by luciferase activities.

FIG. 3a-g. Preservation of stromal cells. a. Representative immunofluorescent images showing proliferating macrophage/microglia (yellow arrowhead) after 2-week and 4-week culture. b. Quantification of CD68+ labeling macrophage/microglia. c. Variable amount of T cells were identified in IPTO based on FACS analysis. d. Immunohistochemistry staining detected CD4+ T cell in brain metastases derived IPTO. e. Immunohistochemistry staining showed CD8+ T cell (red arrow) in brain metastases derived IPTO. f. H&E staining revealed vascular structures (red circle) can be preserved in IPTO. g. Endothelial cells, marked by CD31/CD34, were found in both parental tumor tissue and derived IPTO.

FIG. 4a-b. Therapeutic responses in IPTO. a. Evaluation of drug efficacy by quantifying the proportion of BrdU+ cells to GFP-cells. b. Evaluation of drug toxicity by quantifying the proportion of BrdU+ cells to GFP+ cells.

FIG. 5. Whole genome DNA methylation array was used for molecular classification of human brain tumors. This t-Distributed Stochastic Neighbor Embedding (t-SNE) plot here shows that IPTO (“Liu_IPTO”) clustered very closely with the parental tumor (“Liu_T”).

FIG. 6. Single cell RNA sequencing of IPTO and parental tumor showed that IPTO maintained the entire parental tumor cellular diversity and their molecular identity.

FIG. 7. IPTO chemosensititity predicted patient PFS (progression free survival). The PFS of patients (all received TMZ for treatment) was followed up after testing the TMZ sensitivity of respective IPTOs. The result shows that IPTO predicted TMZ sensitivity and patient response.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

### Individualized Patient-Derived Tumor Organoids (IPTO)

Organoids are three-dimensional cultures of cells that mimic the original tissue. Tumor organoids are a promising tool for disease modeling and drug screening. The existing tumor organoids, however, lack the original tissue architecture (da Silva et al., 2018; Hubert et al., 2016; Linkous et al., 2019), or the interactions between tumor cells and non-tumor cells (Jacob et al., 2019).

Moreover, growing brain tumor organoids is particularly challenging, especially for slow-growing brain tumors, such as glioma with IDH (isocitrate dehydrogenase) mutations and pilocytic astrocytoma.

The instant inventors developed a new individualized patient-derived tumor organoid (IPTO) technology that overcomes various limitations of the conventional tumor organoid technologies. In an example embodiment, a small piece (e.g., 1 mm in diameter) of a tumor tissue is inserted into a host cerebral organoid. The host cerebral organoid is preferably developed from a stem cell, such as an iPSC-derived cerebral organoid. The resulting hybrid organoid can grow for several months. When the tumor piece includes both tumor cells and adjacent stromal cells, both types of cells can be well maintained in the hybrid organoid.

The IPTO has a number of advantages over the existing systems. First, with the IPTO technology, the instant inventors have achieved near 100% success rate in culturing brain tumor organoids. Around 120 patient tumors have been grown into IPTO, including both pediatric and adult glioblastoma, IDH mutant glioma, low grade brain tumors such as pilocytic astrocytoma and oligodendroglioma, and a large variety of brain metastatic tumors (e.g., melanoma, brain lymphoma, lung carcinoma, pancreatic cancer, colorectal carcinoma). Among these, IDH mutant glioma and low-grade brain tumors are known to be particularly difficult to culture.

Mutations in IDH have been found in 70-80% WHO grade 2 or 3 astrocytoma and oligodendrogliomas. The ability to culture slow-growing brain tumor cell, therefore, has tremendous value and was unexpected. It is contemplated that the IPTO benefits from the presence of host cerebral organoids and without the disturbance of tumor tissue architecture.

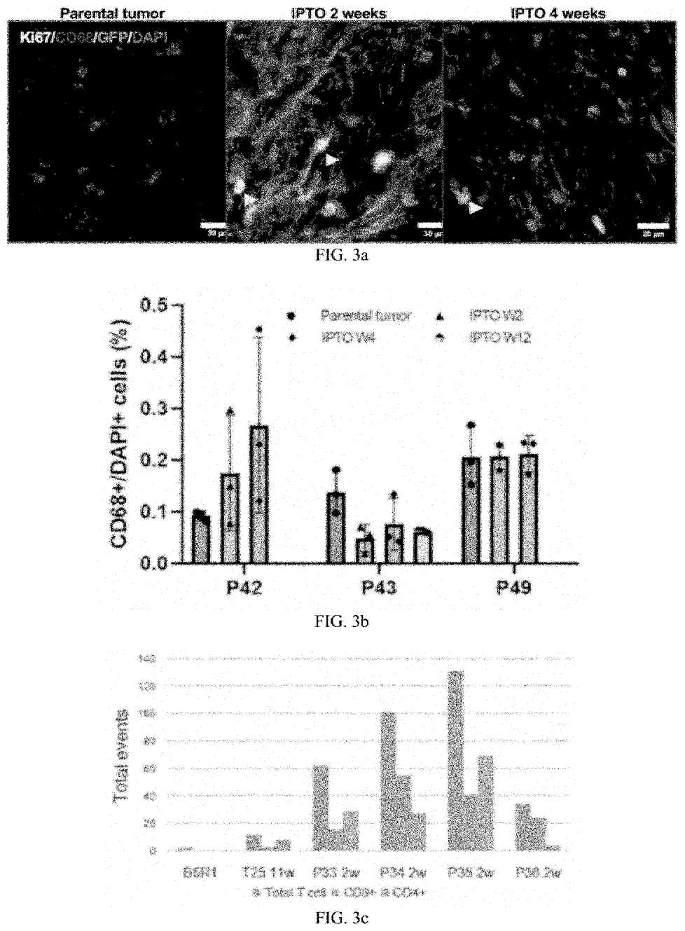

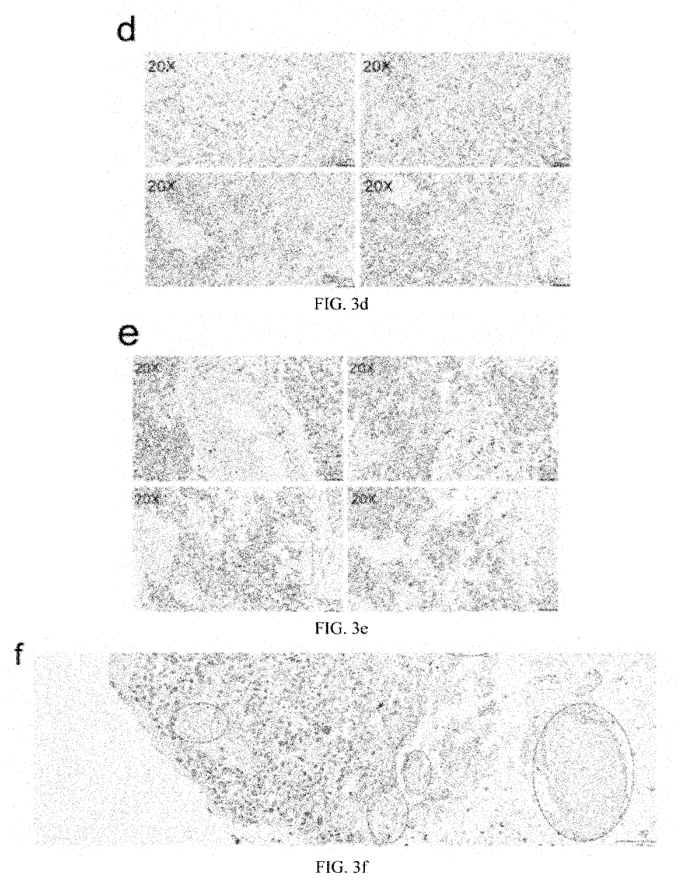

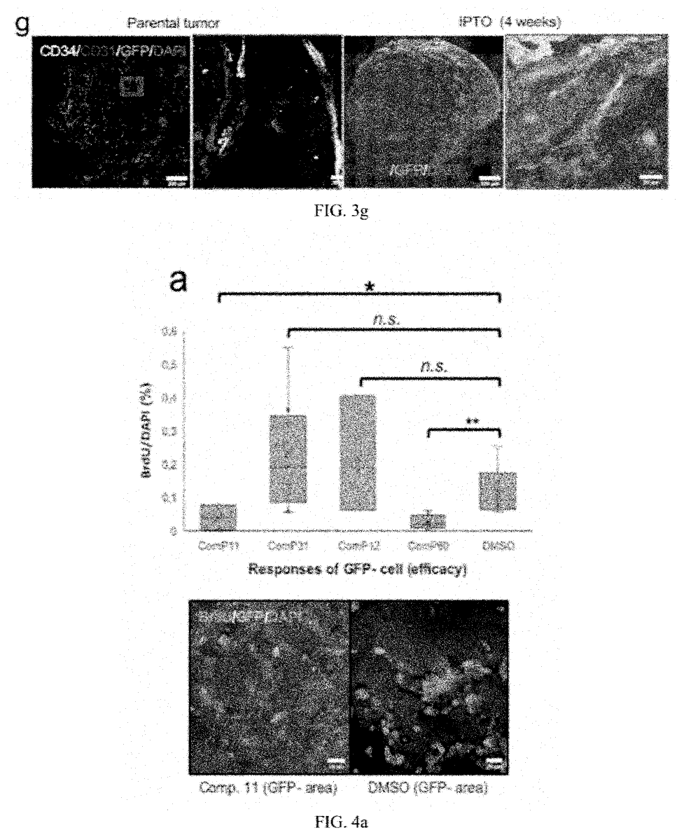

Second, the IPTO can include and preferably includes stromal cells, such as blood vessel, microglial and T cells, which can be grown or maintained in the organoids. In a recently published study culturing glioblastoma tissue (Jacob et al., 2019), the macrophage/microglial cell population suffered from a gradual decrease. In contrast, in the IPTO system, the macrophage/microglia cell population from the majority of samples tested were stable or even enriched (FIG. 3). This is another unexpected finding of the instant disclosure, and a significant advantage. The inclusion of these stromal cells allows testing of therapies such as immunotherapies.

Third, as demonstrated in the experimental examples, the IPTO can be expanded, passaged, cryopreserved and recovered. Also, the preparation, expansion, passaging etc. can be done quite efficiently and quickly. For instance, the entire procedure from surgery to establish organoid can be completed within 2 weeks.

Fourth, as shown in the figures, the cellular and molecular pathology are maintained from patient to organoid, which makes it ideal to test drug sensitivity for patient. Finally, because the culture system harbors heathy host cells, these healthy host cells allow evalation of safety of the candidate drugs.

These and other advantages of the IPTO technology are summarized in Table 1 below.

TABLE 1
Comparisons of in vitro or ex vivo brain tumor models lines

| | 2D cell | | | | | |
|---|---|---|---|---|---|---|
| | lines | Spheres | Organoids | | | |
| | Abbreviation | | | | | |
| | | | | GBO | GLICO | IPTO |
| Source of tumor | PD | PD | PD | PD | GE or PD | PD |
| | GSCs | GSCs | GSCs | tissues | glioma cells | tissues |
| Presence of “normal” | − | − | − | − | + | + |
| tissue | | | | | | |
| Avoid usage of growth | − | − | − | + | Varied | + |
| factors | | | | | | |
| Matrigel | − | − | + | − | Varied | + |
| Efficiency of | 50<% | <50% | NR | 91.40% | Depend on | 100% |
| derivation GBM | | | | | cells | |
| Efficiency of slow- | NR | NR | NR | 66.70% | NR | 100% |
| growing tumors (e.g., | | | | | | |
| IDHmut glioma and | | | | | | |
| Pas) | | | | | | |
| Time of culture | 2-4 | 2-4 | 1-2 | 1-2 | 1-2 | 2 weeks/ |
| establishment | weeks | weeks | months | weeks | months | ready for |
| | | | | | | drug testing |
| Compatibility with | NR | NR | NR | NR | NR | + |
| FF samples | | | | | | |
| Freezing/recovering | + | + | − | + | − | + |
| Maintenance of tumor | − | − | + | + | + | + |
| heterogeneity | | | | | | |
| Tumor/non-tumor | − | − | − | − | + | + |
| interaction | | | | | | |
| Immune cell | − | − | − | + | − | + |
| preservation | | | | | | |
| (microglial, T cells) | | | | | | |
| 3D tissue structure | − | − | + | + | + | + |
| maintenance of genetic | − | − | − | + | − | + |
| mutation spectrum and | | | | | | |
| clonal diversity | | | | | | |
| Maintenance of tumor | − | − | − | + | − | + |
| molecular pathology | | | | | | |
| Treatment toxicity | − | − | − | − | + | + |
| evaluation | | | | | | |
| Tumor entities | GBM | GBM | GBM | GBM | GBM | GBM, HGG, |
| | | | | | | LGG, |
| | | | | | | pediatric |
| | | | | | | brain tumors |
| | | | | | | and all CNS |
| | | | | | | metastasis |
| Correlation with | NA | NA | NA | not | NA | + |
| patient response | | | | demonstrated | | |
| during treatment | | | | | | |

GBM, glioblastoma;
GSC, glioma stem cell;
HTO, hybrid tumor organoid;
IPTO, individualized patient-derived tumor organoid;
NR, not reported;
PAs, pilocytic astrocytoma;
PD, patient-derived,
HGG, High grade glioma,
NA, Not applicable

In accordance with one embodiment of the present disclosure, therefore, provided is a hybrid organoid that includes a tumor tissue embedded in a cerebral organoid.

Cerebral organoids can be prepared with methods known in the art (Lancaster and Knoblich, 2014; Lancaster et al., 2013). An example protocol is provided in Example 1. In some embodiments, the cerebral organoid is differentiated from a stem cell, preferably a pluripotent stem cell, such as an induced pluripotent stem cell (iPSC).

As used herein, “stem cell” defines a cell with the ability to divide for indefinite periods in culture and give rise to specialized cells. Non-limiting examples of types of stem cells include somatic (adult) stem cells, embryonic stem cells, parthenogenetic stem cells (Cibelli et al., 2002; Janus, 2008; Kim, 2010) and/or induced pluripotent stem cells (iPS cells or iPSCs).

As used herein, the term “pluripotent stem cells” refers to cells that are: (i) capable of indefinite proliferation in vitro in an undifferentiated state; (ii) maintain a normal karyotype through prolonged culture; and (iii) maintain the potential to differentiate to derivatives of all three embryonic germ layers (endoderm, mesoderm, and ectoderm) even after prolonged culture. Non-limiting examples of currently available pluripotent stem cells include embryonic stem cells and iPSCs.

The cerebral organoid can be checked and confirmed with known markers, such as neuroepithelial stem cell protein (nestin), doublecortin (DCX), neuron-specific Class III β-tubulin (TuJ1), microtubule associated protein 2 (MAP2), marker of proliferation Ki-67 (KI67), paired box 6 (PAX6), vimentin, and T-box brain transcription factor 1 (TBR1). Nestin is marker for neural stem cells. DCX is a marker for neuroblasts. TuJ1 is a marker for immature neuron. KI67 indicates proliferating neurons. PAX6 indicates forebrain neuronal precursor cells. Phospho-vimentin indicates radial glia. TBR1 indicates deep-layer neurons. MAP2 proteins are neuron-specific cytoskeletal proteins enriched in dendrites and perikarya, implicating a role in determining and stabilizing neuronal morphology during neuron development. In some embodiments, at least two of these markers are detectable in a cerebral organoid. In some embodiments, at least three, four, five, or six of these markers are detectable in a cerebral organoid.

In some embodiments, the cerebral organoid is grown to a size suitable for embedding a tumor tissue. In some embodiments, the suitable size is 0.5-20 mm in diameter, such as 1-15 mm in diameter, 1-10 mm in diameter, 1-8 mm in diameter, 2-6 mm in diameter, or 2-4 mm in diameter, without limitation.

In some embodiments, an incision is made in the cerebral organoid to allow disposition of a tumor piece. In some embodiments, the incision has a depth that is about half to ⅓ of the diameter of the cerebral organoid. In some embodiments, the incision is in about the middle of the cerebral organoid.

In some embodiments, the tumor tissue that is embedded in the cerebral organoid is a tissue of a brain tumor. In some embodiments, the brain tumor is a high-grade brain tumor. In some embodiments, the brain tumor is a low-grade brain tumor. In some embodiments, the brain tumor is a slow-growing brain tumor.

Brain tumors can be classified into four grades. Grade 1 and 2 are also called low-grade tumors. Grade 3 and 4 are also called high-grade. Criteria for clarifying the brain tumors are known in the art. In general, Grade 1 brain tumors are slow growing and unlikely to spread. They can often be cured with surgery; Grade 2 brain tumors are less likely to grow and spread but are more likely to come back after treatment; Grade 3 brain tumors are more likely to have rapidly dividing cells but no dead cells; and Grade 4 brain tumor cells are actively dividing.

In some embodiments, the brain tumor is glioblastoma, pilocytic astrocytoma or oligodendroglioma. In some embodiments, the brain tumor, such as glioma, is characterized with one or more isocitrate dehydrogenase (IDH) mutations. A high percentage of lower grade gliomas harbor mutations in the genes isocitrate dehydrogenase 1 and 2 (IDH1 and IDH2). IDH mutations may be a driver of oncogenesis. Somatic mosaicism for IDH1 or IDH2 at R132 causes the enchondromatosis syndromes, Ollier's disease and Maffucci syndrome, which are characterized by hemangiomas and cartilaginous tumors and which carry an increased risk for gliomas. Also, introduction of mutated IDH into normal cells causes increased proliferation, increased colony formation, and inability to differentiate.

In some embodiments, the brain tumor is a metastatic tumor originated from a different tissue. The tumor from the different tissue, without limitation, may be melanoma, brain lymphoma, bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, prostate cancer and thyroid cancer.

In some embodiments, the tumor embedded in the cerebral organoid has a suitable size. For instance, the tumor tissue has a diameter of 0.2 mm to 5 mm. In some embodiments, the tumor tissue has a diameter of 0.2 to 4 mm, or 0.4 to 3 mm, 0.5 to 2 mm, or 0.8 to 1.5 mm, without limitation.

In some embodiments, the tumor tissue has a diameter that is about 10%-90% of that of the cerebral organoid. In some embodiments, the tumor tissue has a diameter that is about 20%-80% of that of the cerebral organoid. In some embodiments, the tumor tissue has a diameter that is about 25%-75% of that of the cerebral organoid. In some embodiments, the tumor tissue has a diameter that is about 30%-70% of that of the cerebral organoid. In some embodiments, the tumor tissue has a diameter that is about 40%-60% of that of the cerebral organoid.

In some embodiments, the tumor tissue embedded in the cerebral organoid also includes adjacent stromal cells. In some embodiments, the stromal cells include blood vessel cells, microglial cells, and/or T cells.

The hybrid organoids of the present disclosure can be at different stages of growth. In some embodiments, after a period of time of culturing, the number of tumor cells in the hybrid organoid, since embedding into the host cerebral organoid, has increased by at least 20%, 40%, 50%, 100%, 2 folds, 3 folds, 4 folds, 5 folds, 10 folds, or 20 folds.

In some embodiments, after a period of time of culturing, the size of the tumor tissue in the hybrid organoid, since embedding into the host cerebral organoid, has increased by at least 20%, 40%, 50%, 100%, 2 folds, 3 folds, 4 folds, 5 folds, 10 folds, or 20 folds, in diameter.

In some embodiments, the hybrid organoid can be cultured for at least 1 week, 2 weeks, 4 weeks, 2 months, 3 months, 4 months or longer. The culturing may be in a medium such as the modified DMEM medium of Example 2. In some embodiments, the hybrid organoid may be cryopreserved or frozen, and then restored for further use.

In some embodiments, the hybrid organoid expresses one or more markers of platelet endothelial cell adhesion molecule (PECAM-1, CD31), protein tyrosine phosphatase, receptor type, C (PTPRC, CD45)), cluster of differentiation 68 (CD68), doublecortin (DCX), glial fibrillary acidic protein (GFAP), glycerol-3-phosphate dehydrogenase 1 (GPD1), allograft inflammatory factor 1 (Iba1), marker of proliferation Ki-67 (Ki67), microtubule associated protein 2 (MAP2), neuroepithelial stem cell protein (nestin), oligodendrocyte transcription factor (Olig2), S100beta chain, SRY (sex determining region Y)-box 2 (Sox2), and neuron-specific class III β-tubulin (TuJ1). In some embodiments, at least two of these markers are detectable in a cerebral organoid. In some embodiments, at least three, four, five, or six of these markers are detectable in a cerebral organoid.

In one embodiment, the hybrid organoid expresses CD31. In one embodiment, the hybrid organoid expresses CD45. In one embodiment, the hybrid organoid expresses CD68. In one embodiment, the hybrid organoid expresses DCX. In one embodiment, the hybrid organoid expresses GFAP. In one embodiment, the hybrid organoid expresses GPD1. In one embodiment, the hybrid organoid expresses Iba1.

In one embodiment, the hybrid organoid expresses Ki67. In one embodiment, the hybrid organoid expresses MAP2. In one embodiment, the hybrid organoid expresses nestin. In one embodiment, the hybrid organoid expresses Olig2. In one embodiment, the hybrid organoid expresses S100beta chain. In one embodiment, the hybrid organoid expresses Sox2. In one embodiment, the hybrid organoid expresses TuJ1.

### Methods of Using IPTO

The individualized patient-derived tumor organoids (IPTO) prepared according to certain embodiments of the present technology can be used as a model system for various purposes. In some embodiments, the model system can be used to unveil biological mechanisms of tumorigenesis. Also importantly, the model system can be used to evaluate candidate agents potentially useful for treating tumor.

Accordingly, in one embodiment, the present disclosure provides a method for evaluating a candidate agent, which entails contacting a hybrid organoid of the present disclosure with the candidate anticancer agent, and examining effect of the candidate agent on the hybrid organoid. In some embodiments, the tumor tissue embedded in the cerebral organoid also includes adjacent stromal cells. In some embodiments, the stromal cells include blood vessel cells, microglial cells, and/or T cells. Generally, higher death rate of tumor cells indicates better anticancer efficacy and lower death rate of normal cells (e.g., the cells from the host organoid) indicates better drug safety.

As shown in the examples, the tumor cells in an IPTO can grow at high speed. At about 2 weeks after embedding the original tumor piece into the cerebral organoid, the IPTO reaches high tumor cell density, providing a suitable model for drug testing. Accordingly, in some embodiments, the hybrid organoid has been cultured, since the embedding of the tumor tissue in the host cerebral organoid, for about 2 weeks. Alternatively, in some embodiments, the hybrid organoid has been cultured, since embedding of the tumor tissue in the host cerebral organoid, for at least 1 week, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks or 16 weeks. In some embodiments, the hybrid organoid has been cultured, since embedding of the tumor tissue in the host cerebral organoid, for no more than 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks or 16 weeks.

The hybrid organoids of the present disclosure can be at different stages of growth. In some embodiments, after a period of time of culturing, the number of tumor cells in the hybrid organoid, since embedding into the host cerebral organoid, has increased by at least 20%, 40%, 50%, 100%, 2 folds, 3 folds, 4 folds, 5 folds, 10 folds, or 20 folds.

In some embodiments, after a period of time of culturing, the size of the tumor tissue in the hybrid organoid, since embedding into the host cerebral organoid, has increased by at least 20%, 40%, 50%, 100%, 2 folds, 3 folds, 4 folds, 5 folds, 10 folds, or 20 folds, in diameter.

The testing can be customized for a particular tumor or even a particular patient. In some embodiments, the tumor tissue that is embedded in the cerebral organoid is a tissue of a brain tumor. In some embodiments, the brain tumor is a high-grade brain tumor. In some embodiments, the brain tumor is a low-grade brain tumor. In some embodiments, the brain tumor is a slow-growing brain tumor. In some embodiments, the brain tumor is glioblastoma, pilocytic astrocytoma or oligodendroglioma. In some embodiments, the brain tumor, such as glioma, is characterized with one or more isocitrate dehydrogenase (IDH) mutations. In some embodiments, the brain tumor is a metastatic tumor originated from a different tissue. The tumor from the different tissue, without limitation, may be melanoma, brain lymphoma, bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, prostate cancer and thyroid cancer.

The efficacy of the candidate agent as tested herein, therefore, can be specific to the tumor tissue used, or specific to the tumor tissue from a particular patient. Such testing, therefore, provides a ready therapy for the patient.

The IPTO system of the present disclosure is not only useful for testing conventional chemotherapeutic drug, but also for immunotherapies such as PD-L1 and CD47 inhibitors, given the presence of stromal cells including immune cells in the hybrid organoids.

The term immunotherapy refers to the treatment of a tumor by activating or suppressing the immune system, which in turn kills the tumor cells, rather than killing the tumor cells directly. In some embodiments, the immunotherapy is an immune checkpoint inhibitor. In some embodiments, the immunotherapy is an antibody. In some embodiment, the immunotherapy includes a cytokine. In some embodiment, the immunothepray is CAR-T (chimeric antigen receptor-T cell) therapy or a TCR (T cell receptor T cell) therapy.

### Methods of Preparing IPTO

Another embodiments provides a method for preparing a hybrid organoid of the present disclosure. In some embodiments, the method entails disposing a tumor tissue in an incision of a cerebral organoid.

Cerebral organoids can be prepared with methods known in the art (Lancaster and Knoblich, 2014; Lancaster et al., 2013). An example protocol is provided in Example 1. In some embodiments, the cerebral organoid is differentiated from a stem cell, preferably a pluripotent stem cell, such as an induced pluripotent stem cell (iPSC).

The cerebral organoid can be checked and confirmed with known markers, such as neuroepithelial stem cell protein (nestin), doublecortin (DCX), neuron-specific Class III β-tubulin (TuJ1), microtubule associated protein 2 (MAP2), marker of proliferation Ki-67 (KI67), paired box 6 (PAX6), vimentin, and T-box brain transcription factor 1 (TBR1). Nestin is marker for neural stem cells. DCX is a marker for neuroblasts. TuJ1 is a marker for immature neuron. KI67 indicates proliferating neurons. PAX6 indicates forebrain neuronal precursor cells. Phospho-vimentin indicates radial glia. TBR1 indicates deep-layer neurons. MAP2 proteins are neuron-specific cytoskeletal proteins enriched in dendrites and perikarya, implicating a role in determining and stabilizing neuronal morphology during neuron development. In some embodiments, at least two of these markers are detectable in a cerebral organoid. In some embodiments, at least three, four, five, or six of these markers are detectable in a cerebral organoid.

In some embodiments, the cerebral organoid is grown to a size suitable for embedding a tumor tissue. In some embodiments, the suitable size is 0.5-20 mm in diameter, such as 1-15 mm in diameter, 1-10 mm in diameter, 1-8 mm in diameter, 2-6 mm in diameter, or 2-4 mm in diameter, without limitation.

In some embodiments, an incision is made in the cerebral organoid to allow disposition of a tumor piece. In some embodiments, the incision has a depth that is about half to ⅓ of the diameter of the cerebral organoid. In some embodiments, the incision is in about the middle of the cerebral organoid.

In some embodiments, the tumor tissue that is embedded in the cerebral organoid is a tissue of a brain tumor. In some embodiments, the brain tumor is a high-grade brain tumor. In some embodiments, the brain tumor is a low-grade brain tumor. In some embodiments, the brain tumor is a slow-growing brain tumor.

Brain tumors can be classified into four grades. Grade 1 and 2 are also called low-grade tumors. Grade 3 and 4 are also called high-grade. Criteria for clarifying the brain tumors are known in the art. In general, Grade 1 brain tumors are slow growing and unlikely to spread. They can often be cured with surgery; Grade 2 brain tumors are less likely to grow and spread but are more likely to come back after treatment; Grade 3 brain tumors are more likely to have rapidly dividing cells but no dead cells; and Grade 4 brain tumor cells are actively dividing.

In some embodiments, the brain tumor is glioblastoma, pilocytic astrocytoma or oligodendroglioma. In some embodiments, the brain tumor, such as glioma, is characterized with one or more isocitrate dehydrogenase (IDH) mutations. A high percentage of lower grade gliomas harbor mutations in the genes isocitrate dehydrogenase 1 and 2 (IDH1 and IDH2). IDH mutations may be a driver of oncogenesis. Somatic mosaicism for IDH1 or IDH2 at R132 causes the enchondromatosis syndromes, Ollier's disease and Maffucci syndrome, which are characterized by hemangiomas and cartilaginous tumors and which carry an increased risk for gliomas. Also, introduction of mutated IDH into normal cells causes increased proliferation, increased colony formation, and inability to differentiate.

In some embodiments, the brain tumor is a metastatic tumor originated from a different tissue. The tumor from the different tissue, without limitation, may be melanoma, brain lymphoma, bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, prostate cancer and thyroid cancer.

In some embodiments, the tumor embedded in the cerebral organoid has a suitable size. For instance, the tumor tissue has a diameter of 0.2 mm to 5 mm. In some embodiments, the tumor tissue has a diameter of 0.2 to 4 mm, or 0.4 to 3 mm, 0.5 to 2 mm, or 0.8 to 1.5 mm, without limitation.

In some embodiments, the tumor tissue has a diameter that is about 10%-90% of that of the cerebral organoid. In some embodiments, the tumor tissue has a diameter that is about 20%-80% of that of the cerebral organoid. In some embodiments, the tumor tissue has a diameter that is about 25%-75% of that of the cerebral organoid. In some embodiments, the tumor tissue has a diameter that is about 30%-70% of that of the cerebral organoid. In some embodiments, the tumor tissue has a diameter that is about 40%-60% of that of the cerebral organoid.

In some embodiments, the tumor tissue embedded in the cerebral organoid also includes adjacent stromal cells. In some embodiments, the stromal cells include blood vessel cells, microglial cells, and/or T cells.

The hybrid organoids of the present disclosure can be at different stages of growth. In some embodiments, after a period of time of culturing, the number of tumor cells in the hybrid organoid, since embedding into the host cerebral organoid, has increased by at least 20%, 40%, 50%, 100%, 2 folds, 3 folds, 4 folds, 5 folds, 10 folds, or 20 folds.

In some embodiments, after a period of time of culturing, the size of the tumor tissue in the hybrid organoid, since embedding into the host cerebral organoid, has increased by at least 20%, 40%, 50%, 100%, 2 folds, 3 folds, 4 folds, 5 folds, 10 folds, or 20 folds, in diameter.

In some embodiments, the hybrid organoid can be cultured for at least 1 week, 2 weeks, 4 weeks, 2 months, 3 months, 4 months or longer. The culturing may be in a medium such as the modified DMEM medium of Example 2. In some embodiments, the hybrid organoid may be cryopreserved or frozen, and then restored for further use.

In some embodiments, the hybrid organoid expresses one or more markers of platelet endothelial cell adhesion molecule (PECAM-1, CD31), protein tyrosine phosphatase, receptor type, C (PTPRC, CD45)), cluster of differentiation 68 (CD68), doublecortin (DCX), glial fibrillary acidic protein (GFAP), glycerol-3-phosphate dehydrogenase 1 (GPD1), allograft inflammatory factor 1 (Iba1), marker of proliferation Ki-67 (Ki67), microtubule associated protein 2 (MAP2), neuroepithelial stem cell protein (nestin), oligodendrocyte transcription factor (Olig2), S100beta chain, SRY (sex determining region Y)-box 2 (Sox2), and neuron-specific class III β-tubulin (TuJ1). In some embodiments, at least two of these markers are detectable in a cerebral organoid. In some embodiments, at least three, four, five, or six of these markers are detectable in a cerebral organoid.

In one embodiment, the hybrid organoid expresses CD31. In one embodiment, the hybrid organoid expresses CD45. In one embodiment, the hybrid organoid expresses CD68. In one embodiment, the hybrid organoid expresses DCX. In one embodiment, the hybrid organoid expresses GFAP. In one embodiment, the hybrid organoid expresses GPD1. In one embodiment, the hybrid organoid expresses Iba1. In one embodiment, the hybrid organoid expresses Ki67. In one embodiment, the hybrid organoid expresses MAP2. In one embodiment, the hybrid organoid expresses nestin. In one embodiment, the hybrid organoid expresses Olig2. In one embodiment, the hybrid organoid expresses S100beta chain. In one embodiment, the hybrid organoid expresses Sox2. In one embodiment, the hybrid organoid expresses TuJ1.

In some embodiments, the method further includes cryopreserving the hybrid organoid. In some embodiments, the cryopreservation is in a cryopreservation medium that comprises a ROCK inhibitor. In some embodiments, the medium includes 10% DMSO with retinoic acid (RA) and 10 μM of a ROCK inhibitor (e.g., Y-27632).

### EXAMPLES

### Example 1. Preparation of Human Induced Pluripotent Stem Cell (hiPSC)-Derived Cerebral Organoid (CO)

This example illustrates the generation of human induced pluripotent stem cell (hiPSC)-derived cerebral organoid (CO). The procedure was adapted from a previously published method (Lancaster and Knoblich, 2014; Lancaster et al., 2013).

Briefly, on day 0 hiPSCs were dissociated into single cells and seeded in ultra-low attachment 96-well plates at a concentration of 12,000 cells per well, containing stem cell medium supplemented with 4 ng/ml bFGF and 50 μM Rho-associated protein kinase (ROCK) inhibitor. On day 3, the medium was replaced with fresh stem cell medium. From day 5, the organoids were transferred to ultra-low attachment 24-well plates with Neural Induction Medium containing DMEM-F12 supplemented with 1×N2 supplement, 1 μg/ml heparin solution, 1×GlutaMAX and 1×MEM-NEAA, the medium was refreshed every other day. On Day 11, organoids were embedded into droplets of Matrigel and transferred into 6 well plates in NeuroDMEM (50% DMEM-F12, 50% Neurobasal medium, 1×N2, 1×B27), Vitamin A, 2.5 mg/ml Insulin, 0.05 mM BME, 1×GlutaMAX, 1×MEM-NEAA and 1× Penicillin/Streptomycin. On day 15, the medium was replaced with Differentiation Medium consisting of 50% DMEM-F12, 50% Neurobasal medium, 1×N2, 1×B27, 2.5 mg/ml insulin, 0.05 mM BME, 1×GlutaMAX, 1×MEM-NEAA, 1× Penicillin/Streptomycin and continued to be cultured on an orbital shaker. The medium was changed every 2-3 days.

To confirm successful differentiation of hiPSC into the neuronal lineage, the organoids can be fixed, and serial sections can be taken using a cryostat. Sections are stained for markers to confirm cortical and forebrain identity. These can include immunostaining for KI67 (proliferating cells), PAX6 (forebrain neuronal precursor cells), phospho-Vimentin (radial glia), and TBR1 (deep-layer neurons). The presence of KI67, PAX6, phospho-Vimentin, and TBR1 antibody markers can confirm the successful differentiation to early cortical forebrain identity.

Furthermore, distinct spatial locations of each of the markers can reveal the self-organized structured nature of the three-dimensional organoids. For example, the precursor marker PAX6 and post-mitotic neuronal marker TBR1 can be seen as occupying the distinct spatial locations, representing in vitro equivalents of the ventricular zone and cortical plate, respectively.

### Example 2. Preparation of Patient-Derived Hybrid Organoids

This example describes the procedure of co-culturing patient-derived fresh tumor pieces with cerebral organoids, along with passaging, cryopreserving and recovering the individualized patient-derived tumor organoid (IPTO).

### Preparation of Tumor Pieces

Various brain tumors could be cultured with this system, including primary/recurrent GBM, metastatic lymphoma, and metastatic melanoma etc. Suitable tumor tissues are close to tumor border without significant necrosis.

Once the tumor tissues were resected, they were placed in a 15 ml Falcon tube and submerged in PBS. Tumors were then washed by DPBS to get rid of debris and blood cells. To make dissection, tumors were transferred into a 10-cm dish on ice and supplemented with RA+ (retinoic acid+) medium. Next, the tumor bulk was cut with a scalpel into small fragments with a diameter of 1˜2 mm, followed by washing 3-times wash with RA+ medium. The tumor pieces are kept in a modified DMEM medium supplemented with retinoic acid (RA), and containing ROCK inhibitor (RI) Y-27632 (10 μM).

### Preparation of Host Cerebral Organoids (CO)

Host CO can be prepared as described in Example 1. Host CO ranging from 4 weeks to 16 weeks are generally acceptable. A suitable size of the CO is 2-3 mm in diameter. Progressive expansions were observed for the cerebral tissues, and the resulting organoids can shrink after 6-month culture. Our staining showed that immature and mature neuron marks were visible in one-month cerebral organoids, marked by TUJ1 (Neuron-specific Class III β-tubulin) and MAP2 (Microtubule Associated Protein 2), respectively. Similarly, abundant astrocytes existed in cerebral organoids, which expressed GFAP and S100beta for 1-month to 5-month cerebral organoids. Taken together, these organoids harbored diverse neural cell population and are suitable for coculture after 1˜5 months culture.

An incision is made in the middle of the organoids with a scalpel. The incision depth is generally half to ⅓ of the diameter of the organoid. The organoids are transferred to a 15-mL Falcon Tube and washed 3× with DPBS, which is discarded after the organoids sink to the bottom of the tube. The organoid is then transferred to modified DMEM medium with RA and RI.

### Co-Culturing of Tumor Tissues and CO

Organoid embedding sheets were used for the co-culturing. An example commercial product is Catalog #08579 (Stem Cell Technologies, Vancouver, British Columbia, Canada). The COs were transferred onto an embedding sheet and excessive medium was removed. A tumor piece was positioned inside a host CO along the incision using an autoclaved metal toothpick.

30 μl prechilled Matrigel was then added to cover the tumor-organoids hybrids which were preferably positioned centrally. The tumor-organoids hybrids were incubated at 37° C. for 20 min for the Matrigel to solidify. The formed droplets were then rinsed with the modified DMEM, and are cultured stationarily overnight.

The tumor-organoids hybrids, IPTO, were transferred to an orbital shaker, at 75 rpm. The medium was changed 2 days later, and every 2-3 days thereafter.

IPTO prepared with this method were stained positively with antibodies against the following markers, CD31 (Platelet endothelial cell adhesion molecule (PECAM-1)), CD45 (Protein tyrosine phosphatase, receptor type, C (PTPRC)), CD68 (Cluster of Differentiation 68), DCX (Doublecortin), GFAP (Glial fibrillary acidic protein), GPD1 (glycerol-3-phosphate dehydrogenase 1), Iba1 (Allograft inflammatory factor 1), Ki67 (Marker Of Proliferation Ki-67), MAP2 (microtubule associated protein 2), Nestin, Olig2 (Oligodendrocyte transcription factor), S100beta chain, Sox2 (SRY (sex determining region Y)-box 2), and TuJ1 (Neuron-specific Class III β-tubulin).

### Passaging of the Hybrid Organoids

IPTO can be passaged to avoid necrosis. “Mature IPTO” as used here refer to those having acceptable organoid size and tumor cell proportion. For instance, the IPTO size (diameter) should be larger than 3-4 mm, or the percentage of tumor cells in IPTO is higher than 50%.

The time for passaging could range from 4 weeks to 16 weeks, depends on how fast the tumor cells grow. The IPTOs are cut into smaller pieces (e.g., about 1 mm in diameter) and cultured in similar fashion as the original IPTOs.

With 2˜3 months culture, the tumor cells dominated the organoids as well as the transparent margin of matrigel. This required passaging to ensure enough oxygen and gradients. IPTOs could be cultured for as long as 68 weeks, where considerable Ki67+ proportion could be observed. Staining showed that tumor markers (GFAP for GBM) were abundant and stable in first 3 months culture. Interestingly, the expression of Nestin or SOX2 was quite stable or enriched over time.

The mature IPTOs can be cryopreserved, and then recovered prior to use.

### Cryopreservation and Recovering Tumor Samples or Tumor in IPTO

Samples from surgical tumors or IPTO could be cryopreserved. For both sample sources, samples should be first dissected in appropriate size on ice. After being washed with DPBS/RA+ medium, sample pieces were placed on orbital shaker (75 rpm, 37° C., 5% CO₂) in RA+ medium supplemented with 10 μM Rock Inhibitor for one hour. Cryopreservation medium was added for another 10 min to equilibrate them. Cryopreservation medium were comprised of RA+ medium, 10% DMSO and 20 μM Rock inhibitor. Finally, samples pieces were transferred into a cryotube in a CoolCell freezing container at −80° C. overnight. For long-term storage, samples were further transferred to be kept in liquid nitrogen.

To recover samples, cryovials were first thawed in a 37° C. water bath until only a small chunk of ice remains visible. 1 mL RA+ with 10 μM RI were added into a 6-well plate and gently transferred samples into the plate using a P1000 pipette with a cut tip. Afterwards, the medium containing DMSO was discarded and replaced with fresh RA+ with 10 μM RI. Thereafter, these recovered samples were ready to be co-cultureed with fresh cerebral organoids, as depicted in Generation of IPTO.

The following four cryopreservation medium were tested to explore an optimal working regimen: Group 1 (RA+ plus 10% DMSO), Group 2 (RA+ plus 10% DMSO plus 10 μM RI), Group 3 (RA+ plus 10% DMSO plus 10 μM RI plus 0.5M. Trehalose), and Group 4 (FBS plus 10% DMSO and 10 μM RI). Cerebral organoids were introduced from iPSC clones that stably expressed firefly luciferase. Consequently, BL results showed that the signal of Group 1 tended to be lower at Day 3, but increased later and comparable to Group 2, Group 3, and Group 4. But the Ki67 proportion in Group 1 tended to be lower than the other three groups. This indicated the necessity to add 10 μM RI in cryopreservation medium. The Ki67 proportion in Group 2 was higher than that in Group 3, suggesting 0.5M Trehalose did not generate additional benefits. This was consistent to earlier findings that >0.1M trehalose significantly increased osmotic pressure, which might counteract benefits of reducing the ice crystals by adding this sugar. Hence, RA+ plus 10% DMSO plus 10 μM RI was used to cryopreserve parental tissues or organoid tissues. Comparable histological features and Ki67 staining could be achieved. In addition, microglia cells also survived in IPTOs recovered from cryopreserved tumor tissues

### Infection of Tumor in IPTO and Bioluminescence Imaging

To allow only cells from tumor samples to express firefly luciferase, tumor fragments were infected with lentivirus before coculturing. Accordingly, the processed tumor fragments were treated with lentivirus at the optimized concentration overnight. Polybrene (Merck Millipore, #TR-1003-50UL) was used to increase the infection efficiency. Coculture procedure was conducted the next day as aforementioned. For Bioluminescence imaging, IPTOs were refreshed with RA+ medium containing 150 μg/ml D-luciferin (BioVision, #7903), followed by being incubated for 10 minutes on orbital shaker at 37 C. The bioluminescence images (BLIs) were captured with the IVIS Lumina II (Perkin Elmer) with an exposure time of 60 seconds and 5 consecutive segments. The maximal total pixels among the five segments were chosen for analysis.

### Example 3. Preparation of Individualized Patient-Derived Tumor Organoid (IPTO) and Use for Drug Evaluation

This example used the process described in Examples 1 and 2 to prepare tumor hybrid organoids, which were then used to test the efficiency of anticancer agents.

hiPSC-derived cerebral organoids (COs) were prepared as described in Example 1. In addition, the cells were genetically engineered to express green fluorescent protein (GFP) for visualization.

Brain tumor tissues were obtained from patients having IDH (isocitrate dehydrogenase)-mutant glioma and pilocytic astrocytoma. The tissues were cut into species and cocultured with the hiPSC-derived COs, as described in Example 2 (FIG. 1a). At 4 weeks old, the host organoids were used for co-culture with tumor tissues. At this time point, both the normal and B2M (beta 2-microglobulin)-knock out organoid expressed markers for neural stem cells (nestin), neuroblast (DCX) and immature neuron (TUJ1) (FIG. 1b).

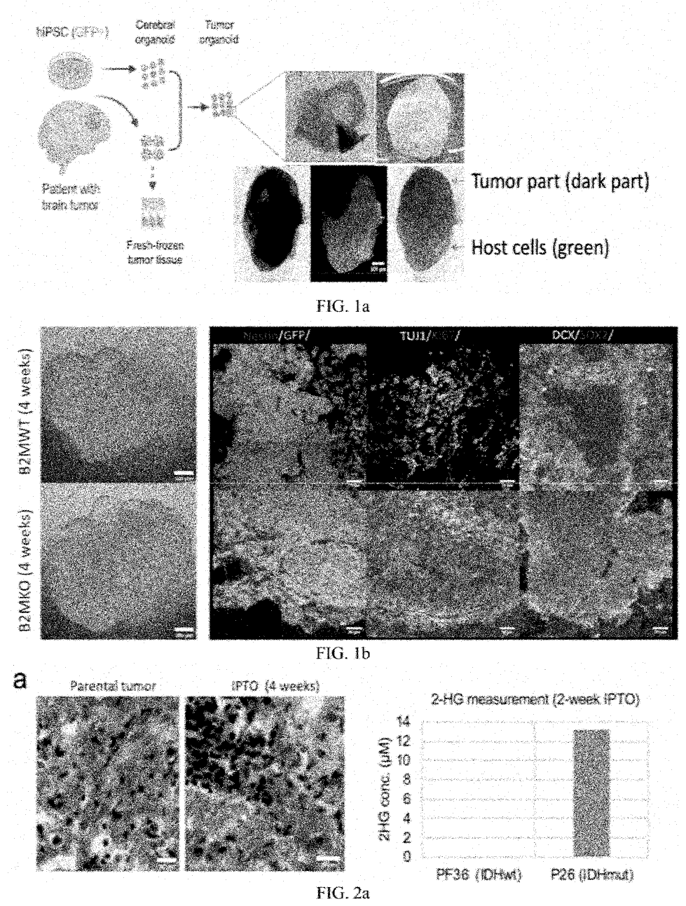

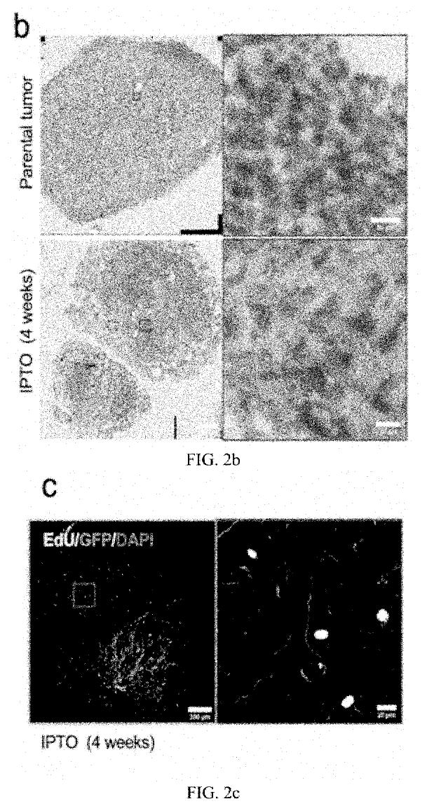

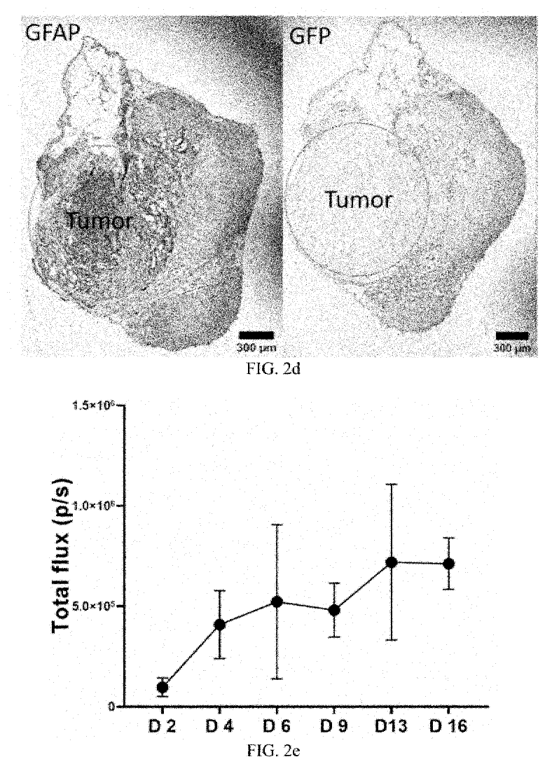

As shown in FIG. 2a, the cell density of IDHmut glioma-derived tumor organoids (IPTO) was comparable to that of the parental tumor (H&E staining, left) and secretion of 2-HG was detectable. Representative IHC (IDHR132H) images showed spatial distribution of IDHmut glioma cells in the hybrid tumor organoids (FIG. 2b). As shown in FIG. 2d, the expression of glial fibrillary acidic protein (GFAP), which is a marker for glioblastoma, was very high in the tumor part. Meanwhile, the tumor area was also indicated by lack of GFP expression (FIG. 2d). Likewise, tumor cell density in pilocytic astrocytoma-derived organoids was high as well, and proliferating cells in the tumor area (GFP-) were identified (FIG. 2c).

Most of the IPTOs reached high cell population (indicated by bioluminescence signal intensity) at around two weeks after the co-culture. Therefore, IPTOs at about 2 weeks are considered as suitable for drug testing.

Culturing of brain tumors has proven to be challenging. Moreover, there are no models for brain tumors with mutations in IDH, which were found in 70-80% WHO grade 2 or 3 astrocytomas and oligodendrogliomas. Therefore, the observation of IDHmut glioma being cultured in hybrid tumor organoids as evidenced by the high tumor cell density and 2-hydroxyglutarate (HG) secretion (FIG. 2b) was unexpected.

Also, compared with high-grade glioma, there were few authenticated preclinical models for pilocytic astrocytoma. Encouragingly, in this example, pilocytic astrocytomas survived in their derived IPTOs (FIG. 2c). Therefore, IPTO showed superiority to culture brain tumors, including slow-growing ones.

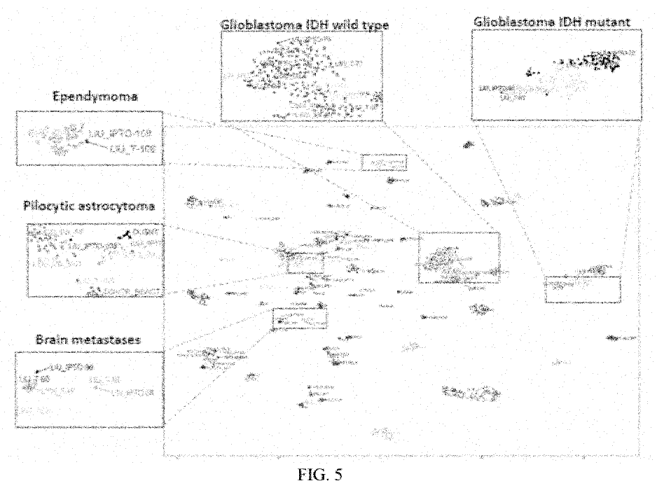

DNA methylation profiles have been shown to lead to diagnostic precision compared to standard methods. In order to investigate whether IPTO could recapitulate molecular pathology of their original tumor, DNA methylation analysis was conducted and findings revealed that epigenetics signatures of glioblastoma-derived IPTO resembled their corresponding parental tumor. Whole genome DNA methylation array was used for molecular classification of human brain tumors, which is a new standard for WHO 2021 brain tumor classification. FIG. 5 shows a t-Distributed Stochastic Neighbor Embedding (t-SNE) plot showing that similar tumors clustered together. Each spot represents a sample (parental tumor or IPTO). As shown in the figure, in particular the blown-up ones, IPTO clustered very closely with the parental tumors.

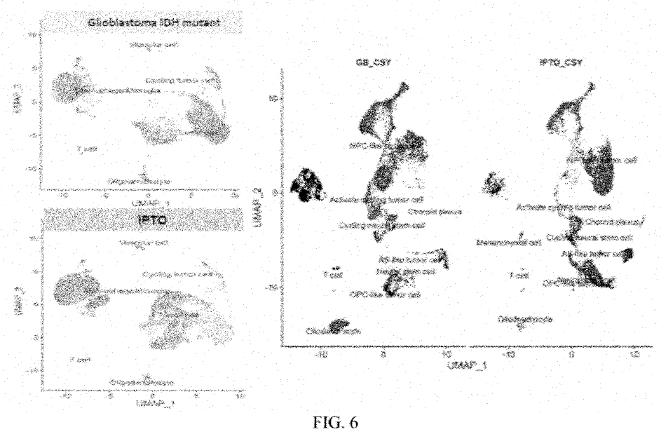

Moreover, copy number variations (CNVs) in brain metastases derived IPTO were almost identical to their original tumors. Likewise, when single-cell RNA sequencing was conducted, the results (FIG. 6) show that IPTO samples co-clustered with the parental tumors, indicating that the IPTO preserved the tumor microenviorment of the parental tumors.

In a recently published study directly culturing glioblastoma tissue alone (Jacob et al., 2019), various stromal cells were maintained in their patient-derived organoids (PDO). However, the macrophage/microglial cell population suffered from a gradual decrease. In contrast, IPTO benefited from the supports from host organoids and macrophage/microglia cell population from the majority of samples were stable or even enriched (FIG. 3a-b). Notably, tumor residual T cells were detectable when tumors were cultured in IPTO (FIG. 3c-e). Moreover, microvascular structures (FIG. 3f) and endothelial cells (FIG. 3g) were preserved in the derived IPTO.

The preservation of 3-D tissue architecture and the cross-talks between non-tumor and tumor cells are believed to better recapitulate brain tumor niche and thus better support tumor and stromal cell growth. The superior recapitulation of tumor organoids in the tumor ecosystem should lead to precise therapeutic responses. The IPTO prepared here were then used to test the efficacy and safety of candidate drugs.

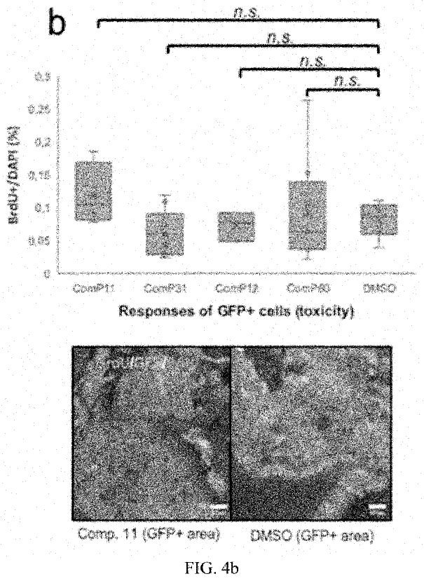

Four compounds, ComP11, ComP31, ComP12 and ComP60, were added to the IPTO and their impact on cell viability was evaluated with BrdU staining. Drug efficacy was quantitated by the proportion of BrdU+ cells to GFP-cells (tumor cells, FIG. 4a), and drug safety was quantitated by the proportion of BrdU+ cells to GFP+ cells (normal cells, FIG. 4b). ComP11 and ComP60 appeared to be both efficacious and safe as evaluated by these IPTO.

### Example 4. Drug Sensitivity Testing with IPTO and Clinical Validation

This example used the IPTO model and drug screening procedure exemplified in Example 3 to select a drug and validated the drug in human patients.

Before drug testing, IPTOs were transferred to 24-well plates, one IPTO per well, 3-6 IPTOs were randomly selected as a group for drug testing. The viability of IPTOs was monitored by Bioluminescence Imaging using the Quick View 3000 Imaging System (Bio-Real Sciences). The expressed luciferase in the IPTOs convert the substrate D-luciferin into oxyluciferin in an oxygen and ATP-dependent process, leading to the emission of photons. D-luciferin were added into culture medium of IPTOs at a concentration of 150 μg/ml. After 15 minutes, the photons emitted from the IPTOs were monitored by the Quick View 3000 Imaging System, the total signal was calculated in photon/see (p/s).

After signal monitoring, the average signal of IPTOs in the drug test group was normalized to the average signal of the control group, the fold change of the signal at the end point of the drug test relative to the signal at the beginning is then calculated. If the signal was continuously decreasing and the fold change is less than 1, the IPTOs were considered sensitive to the drug; and if the signal was increasing or unchanged and the fold change was greater than 1, the IPTOs were considered insensitive to the drug.

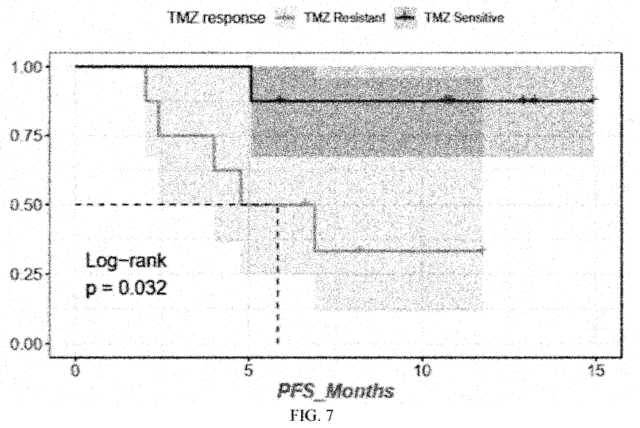

In this test, the IPTOs prepared from some glioblastoma patients were determined to be sensitive to temozolomide (TMZ) while others were determined to be insensitive. All of the patients were treated with TMZ. The follow-up time was 15 months. As shown in FIG. 7, patients that were determined as sensitive to TMZ (TMZ Sensitive) had signifcantly higher progress-free survival (PFS) than those that were determined as insensitive to TMZ (TMZ Resistant). These results, therefore, confirmed the accuracy of the IPTO for testing a patient's sensitivity to potential therapies.

### REFERENCES

Cibelli, J. B., Grant, K. A., Chapman, K. B., Cunniff, K., Worst, T., Green, H. L., Walker, S. J., Gutin, P. H., Vilner, L., Tabar, V., et al. (2002). Parthenogenetic stem cells in nonhuman primates. Science (New York, N.Y.) 295, 819. 10.1126/science.1065637.
da Silva, B., Mathew, R. K., Polson, E. S., Williams, J., and Wurdak, H. (2018). Spontaneous Glioblastoma Spheroid Infiltration of Early-Stage Cerebral Organoids Models Brain Tumor Invasion. SLAS discovery: advancing life sciences R & D 23, 862-868. 10.1177/2472555218764623.
Hubert, C. G., Rivera, M., Spangler, L. C., Wu, Q., Mack, S. C., Prager, B. C., Couce, M., Mclendon, R. E., Sloan, A. E., and Rich, J. N. (2016). A Three-Dimensional Organoid Culture System Derived from Human Glioblastomas Recapitulates the Hypoxic Gradients and Cancer Stem Cell Heterogeneity of Tumors Found In Vivo. Cancer research 76, 2465-2477. 10.1158/0008-5472.can-15-2402.
Jacob, F., Salinas, R. D., Zhang, D. Y., Nguyen, P. T. T., Schnoll, J. G., Wong, S. Z. H., Thokala, R., Sheikh, S., Saxena, D., Prokop, S., et al. (2019). A Patient-Derived Glioblastoma Organoid Model and Biobank Recapitulates Inter- and Intra-tumoral Heterogeneity. Cell. 10.1016/j.cell.2019.11.036.
Janus, E. S. R. L. N. K. N. A. T. J. D. (2008). Patient-specific stem cell lines derived from human parthenogenetic blastocysts.
Kim, K. D., G (2010). Methods for producing embryonic stem cells from parthenogenetic embryos. patent application Ser. No. 12/440,964
Lancaster, M. A., and Knoblich, J. A. (2014). Generation of cerebral organoids from human pluripotent stem cells. Nat Protoc 9, 2329-2340. 10.1038/nprot.2014.158.
Lancaster, M. A., Renner, M., Martin, C. A., Wenzel, D., Bicknell, L. S., Hurles, M. E., Homfray, T., Penninger, J. M., Jackson, A. P., and Knoblich, J. A. (2013). Cerebral organoids model human brain development and microcephaly. Nature 501, 373-379. 10.1038/nature12517.
Linkous, A., Balamatsias, D., Snuderl, M., Edwards, L., Miyaguchi, K., Milner, T., Reich, B., Cohen-Gould, L., Storaska, A., Nakayama, Y., et al. (2019). Modeling Patient-Derived Glioblastoma with Cerebral Organoids. Cell reports 26, 3203-3211 e3205. 10.1016/j.celrep.2019.02.063.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A hybrid organoid, comprising a tumor tissue embedded in a cerebral organoid.

2. The hybrid organoid of claim 1, wherein the cerebral organoid is differentiated from a stem cell.

3. The hybrid organoid of claim 1, wherein the stem cell is an induced pluripotent stem cell (iPSC).

4. The hybrid organoid of claim 1, wherein the cerebral organoid expresses at least a marker selected from the group consisting of neuroepithelial stem cell protein (nestin), doublecortin (DCX), neuron-specific Class III β-tubulin (TuJ1), microtubule associated protein 2 (MAP2), marker of proliferation Ki-67 (KI67), paired box 6 (PAX6), vimentin, and T-box brain transcription factor 1 (TBR1).

5. The hybrid organoid of claim 1, wherein the tumor tissue has a size of 0.2 mm to 5 mm in diameter when disposed in the cerebral organoid.

6. The hybrid organoid of claim 1, wherein the tumor tissue has a size of 0.5 mm to 2 mm in diameter when disposed in the cerebral organoid.

7. The hybrid organoid of claim 1, wherein the tumor tissue is a tissue of brain tumor.

8. The hybrid organoid of claim 7, wherein the brain tumor is selected from the group consisting of glioblastoma, pilocytic astrocytoma, oligodendroglioma, and a metastatic tumor originated from another tissue.

9. The hybrid organoid of claim 8, wherein some of the brain tumor is characterized with a mutation in an isocitrate dehydrogenase (IDH).

10. The hybrid organoid of claim 1, wherein the tumor tissue comprises tumor cells and adjacent non-tumor stromal cells.

11. The hybrid organoid of any preceding-claim 1, wherein the tumor tissue has grown at least 50% in tumor cell number as compared to when the tumor tissue was initially disposed in the cerebral organoid.

12. The hybrid organoid of claim 1, wherein the tumor tissue has grown at least 2-fold in tumor cell number as compared to when the tumor tissue was initially disposed in the cerebral organoid.

13. The hybrid organoid of claim 1, wherein the tumor tissue has at least doubled in size as compared to when the tumor tissue was initially disposed in the cerebral organoid.

14. The hybrid organoid of claim 1, which expresses at least a marker selected from the group consisting platelet endothelial cell adhesion molecule (PECAM-1, CD31), protein tyrosine phosphatase, receptor type, C (PTPRC, CD45)), cluster of differentiation 68 (CD68), doublecortin (DCX), glial fibrillary acidic protein (GFAP), glycerol-3-phosphate dehydrogenase 1 (GPD1), allograft inflammatory factor 1 (Iba1), marker of proliferation Ki-67 (Ki67), microtubule associated protein 2 (MAP2), neuroepithelial stem cell protein (nestin), oligodendrocyte transcription factor (Olig2), S100beta chain, SRY (sex determining region Y)-box 2 (Sox2), and neuron-specific class III β-tubulin (TuJ1).

15. A method of evaluating a candidate anticancer agent, comprising contacting the hybrid organoid of claim 1 with the candidate anticancer agent, and examining change of cell numbers in the hybrid organoid.

16-20. (canceled)

21. A method for preparing a hybrid organoid of claim 1, comprising disposing a tumor tissue in an incision of a cerebral organoid.

22. The method of claim 21, further comprising covering the hybrid organoid with Matrigel® solubilized basement membrane matrix, and allowing the matrix to solidify.

23. The method of claim 21, wherein the tumor tissue has a size of 0.2 mm to 5 mm in diameter when disposed in the cerebral organoid.

24. (canceled)

25. The method of claim 21, wherein the tumor tissue is a tissue of brain tumor.

26. (canceled)

27. The method of claim 21, wherein the cerebral organoid is prepared from an induced pluripotent stem cell (iPSC).

28-30. (canceled)
